(19) United States
(12) Patent Application Publication
(10) Pub. No.: US 2024/0074981 A1
(43) Pub. Date: Mar. 7, 2024

(54) NON-VIRAL DELIVERY OF SMALL MOLECULE THERAPEUTICS

(71) Applicant: BATTELLE MEMORIAL INSTITUTE, Columbus, OH (US)

(72) Inventors: Cherry Gupta, Columbus, OH (US); Julianne N.P. Smith, Dublin, OH (US); Miguel D. Pedrozo, Columbus, OH (US); Nickolas R. Andrioff, Columbus, OH (US); Morris O. Makobongo, Lewis Center, OH (US); Anthony D. Duong, Columbus, OH (US); Michael S. Koeris, Columbus, OH (US)

(21) Appl. No.: 18/332,101

(22) Filed: Jun. 9, 2023

Related U.S. Application Data
(60) Provisional application No. 63/350,650, filed on Jun. 9, 2022.

(51) Int. Cl. A61K 9/50; A61K 31/69; A61K 47/54; A61P 35/00

(52) U.S. Cl. CPC A61K 9/5031; A61K 31/69; A61K 47/549; A61P 35/00

(57) ABSTRACT

The invention relates to nucleic acid nanostructure delivery compositions for non-viral delivery, and methods therefor. More particularly, the invention relates to nucleic acid nanostructure delivery compositions, such as DNA origami compositions, for the delivery, for example, of small molecule therapeutics, and methods therefor.

### CROSS-REFERENCE TO RELATED APPLICATIONS

This non-provisional application claims the benefit and priority, under 35 U.S.C. § 119(e) and any other applicable laws and statutes, to U.S. Provisional Application Ser. No. 63/350,650 filed on Jun. 9, 2022, the entire disclosure of which is incorporated herein by reference.

### FIELD OF THE DISCLOSURE

The invention relates to nucleic acid nanostructure delivery compositions for non-viral delivery of small molecule therapeutics, and methods therefor. More particularly, the invention relates to nucleic acid nanostructure delivery compositions, such as DNA origami compositions, for the delivery, for example, of small molecule therapeutics, and methods therefor.

### BACKGROUND AND SUMMARY

The efficacy of frontline cancer therapies, such as therapies that utilize small molecule therapeutics, is limited by severe toxicity and inadequate generation of anti-tumor immunity critical for long-term survival of patients. The mammalian immune system provides a means for the recognition and elimination of cancer cells and other pathogenic cells. While the immune system normally provides a strong line of defense, there are many instances where cancer cells evade a host immune response and proliferate or persist with concomitant host pathogenicity. Chemotherapeutic agents and radiation therapies have been developed to eliminate, for example, replicating cancers. However, many of the currently available chemotherapeutic agents and radiation therapy regimens have adverse side effects because they work not only to destroy cancers, but they also affect normal host cells, such as cells of the hematopoietic system. The adverse side effects of these anticancer drugs highlight the need for the development of new therapies selective for cancers with reduced host toxicity and with the ability to generate anti-tumor immunity. Additionally, cancer cells may develop apoptosis resistance mechanisms, decreasing their sensitivity to conventional chemotherapeutic agents that induce apoptotic cell death.

Researchers have developed therapeutic protocols for destroying cancers by targeting cytotoxic compounds to cancer cells, such as specific cancer cells. Many of these protocols utilize toxins conjugated to antibodies that bind to antigens unique to or overexpressed by the cancer cells in an attempt to minimize delivery of the toxin to normal cells. Another approach for targeting cancer cells in a host is to enhance the host immune response against the cancer cells to avoid the need for administration of compounds that may exhibit independent host toxicity and to confer long-term cancer protection mediated by anti-tumor adaptive immunity. Another strategy is to target immunosuppressive cells such as tumor-associated macrophages (TAMs) or myeloid-derived suppressor cells (MDSCs) to enhance the immune response against cancer cells.

The potential for non-apoptotic, immunogenic programmed cell death (PCD) to promote anti-tumor immunity through the activation of innate and adaptive immune cells has recently been recognized. As one example, the small molecule talabostat (TLBST) induces a highly inflammatory form of PCD known as pyroptosis. Unlike apoptosis, pyroptosis involves inflammasome activation, caspase 1-mediated IL-1β and IL-18 maturation, lytic cell death, and release of intracellular contents. Pyroptosis was first identified in myeloid cells, but cancer cells can also undergo pyroptosis. In murine cancer models, systemic administration of TLBST demonstrated potent T and NK cell-dependent protection, yet efficacy in phase two clinical trials varied, due to inefficient TLBST uptake by cancer cells and systemic toxicity.

The inventors have developed nucleic acid nanostructure delivery compositions (e.g., DNA origami structures) for the delivery of small molecule therapeutics to cells, including cancer cells and immunosuppressive cells such as TAMs and MDSCs. Relative to other nanocarriers, nucleic acid nanostructure delivery compositions (e.g., DNA origami structures) can be precisely programmed for shape, size and functionality, form uniquely homogeneous populations, and are highly biocompatible. The current state-of-the-art non-viral gene delivery systems, such as liposomes, have many drawbacks such as poor biocompatibility and the inability to easily engineer or functionalize them. The nucleic acid nanostructure delivery compositions (e.g., DNA origami nanostructures) developed by the inventors have the advantage of being biocompatible, non-toxic, and can be programmed in many ways. For example, the nucleic acid nanostructure delivery compositions can be programmed to have functional groups that enable them to evade early degradation, that enable them to evade immune responses, and that enable targeted and controlled delivery of small molecule therapeutics. Thus, these non-viral delivery compositions can enhance the stability, safety, and/or efficacy of small molecule therapeutics by providing immune evasion and tissue-directed intracellular delivery, and by providing the potential to enhance anti-tumor immunity.

The following clauses, and combinations thereof, provide various additional illustrative aspects of the invention described herein. The various embodiments described in any other section of this patent application, including the section titled “DETAILED DESCRIPTION OF ILLUSTRATIVE EMBODIMENTS” and the “EXAMPLES” are applicable to any of the following embodiments of the invention described in the numbered clauses below.

1. A composition comprising a non-viral delivery vehicle comprising a nucleic acid nanostructure delivery composition, and a small molecule therapeutic.

2. The composition of clause 1, wherein the nucleic acid nanostructure delivery composition comprises a DNA origami composition.

3. The composition of clause 1, wherein the nucleic acid nanostructure delivery composition comprises single-stranded or double-stranded DNA or RNA.

4. The composition of clause 3, wherein the nucleic acid nanostructure delivery composition comprises DNA.

5. The composition of clause 3, wherein the nucleic acid nanostructure delivery composition comprises RNA.

6. The composition of clause 3 wherein the nucleic acid nanostructure delivery composition comprises RNA and DNA.

7. The composition of any one of clauses 3 and 4 to 6, wherein the nucleic acid nanostructure delivery composition comprises both single-stranded and double-stranded regions of the nucleic acids.

8. The composition of any one of clauses 3 to 6, wherein the nucleic acid nanostructure delivery composition is single-stranded.

9. The composition of any one of clauses 3 to 6, wherein the nucleic acid nanostructure delivery composition is double-stranded.

10. The composition of any one of clauses 1 to 9, wherein the small molecule therapeutic is associated with the nucleic acid nanostructure delivery composition by a high affinity, non-covalent bond interaction between a biotin molecule on the small molecule therapeutic and a molecule that binds to biotin on the nucleic acid nanostructure delivery composition.

11. The composition of clause 10, wherein the molecule that binds to biotin is bound to the nucleic acid nanostructure delivery composition by a covalent phosphonamidite bond formed via an EDC-NHS coupling reaction between a terminal phosphate group of a 5′ end of an overhang on the nucleic acid nanostructure delivery composition and an amine group on the molecule that binds to biotin.

12. The composition of clause 10 or 11, wherein the biotin is bound to the small molecule therapeutic by a covalent bond.

13. The composition of any one of clauses 1 to 9, wherein the small molecule therapeutic is bound to the nucleic acid nanostructure delivery composition by a covalent bond.

14. The composition of clause 13, wherein the covalent bond is formed via an EDC-NHS coupling reaction between a terminal phosphate group of the 5′ end of an overhang on the nucleic acid nanostructure delivery composition and an amine group on the small molecule therapeutic.

15. The composition of clause 13, wherein the covalent bond is formed via a click chemistry coupling reaction between an azide group on the nucleic acid nanostructure delivery composition and an alkyne group on the small molecule therapeutic.

16. The composition of clause 13, wherein the covalent bond is formed via a click chemistry coupling reaction between an azide group on the small molecule therapeutic and an alkyne group on the nucleic acid nanostructure delivery composition.

17. The composition of any one of clauses 1 to 9, wherein the small molecule therapeutic is associated with the nucleic acid nanostructure delivery composition by a covalent bond between a carboxy terminated molecule on the nucleic acid nanostructure delivery composition and a primary amine on the small molecule therapeutic.

18. The composition of any one of clauses 1 to 9, wherein the small molecule therapeutic is associated with the nucleic acid nanostructure delivery composition by an electrostatic interaction between a negatively charged nucleic acid nanostructure delivery composition and a positively charged amine in the small molecule therapeutic.

19. The composition of any one of clauses 1 to 9, wherein the small molecule therapeutic is associated with the nucleic acid nanostructure delivery composition by intercalation of the small molecule therapeutic into the nucleic acid nanostructure delivery composition.

20. The composition of any one of clauses 1 to 19, wherein the small molecule therapeutic causes pyroptosis.

21. The composition of any one of clauses 1 to 19, wherein the small molecule therapeutic causes apoptosis.

22. The composition of any one of clauses 1 to 19, wherein the small molecule therapeutic causes necroptosis.

23. The composition of any one of clauses 1 to 18 and 20, wherein the small molecule therapeutic is a post-proline cleaving enzyme inhibitor.

24. The composition of clause 23, wherein the small molecule therapeutic inhibits a post-proline cleaving dipeptidyl peptidase (DPP) selected from DPP4, DPP8, DPP9, and fibroblast activation protein.

25. The composition of any one of clauses 1 to 24, wherein the small molecule therapeutic induces anti-tumor immunity.

26. The composition of any one of clauses 1 to 25, wherein the small molecule therapeutic induces cytokine production.

27. The composition of any one of clauses 1 to 26, wherein the small molecule therapeutic induces inflammasome activation.

28. The composition of clause 26, wherein the small molecule therapeutic induces the production of an interferon or an interleukin.

29. The composition of clause 28, wherein the interferon and the interleukin are selected from a type one interferon, IFN-β, IFN-γ, IL-1β, IL-6, IL-12p70, and IL-18.

30. The composition of clause 26, wherein the cytokine is selected from TNF-α and MCP-1/CCL2.

31. The composition of any one of clauses 1 to 30, wherein the small molecule therapeutic causes cancer cell lysis.

32. The composition of any one of clauses 1 to 18, 20, and 23 to 31, wherein the small molecule therapeutic is talabostat.

33. The composition of any one of clauses 1 to 32, wherein the nucleic acid nanostructure delivery vehicle comprises a cell-targeting molecule.

34. The composition of clause 33, wherein the cell-targeting molecule is selected from an antibody, an aptamer, a peptide, PNA, and a small molecule cell-targeting molecule.

35. The composition of clause 34, wherein the cell-targeting molecule is IL4Pep1.

36. The composition of any one of clauses 1 to 35, wherein the nucleic acid nanostructure delivery composition is coated with one or more polymers.

37. The composition of clause 36, wherein the one or more polymers comprise PEG-poly-L-lysine.

38. The composition of clause 32, wherein the talabostat modulates the activity of a molecule selected from DPP, NLRP1, CARD8, and a gasdermin family member.

39. A method of treating a patient with a disease, the method comprising administering to the patient any of the compositions of clauses 1 to 38 or clause 68, and treating the disease in the patient.

40. The method of clause 39, further comprising administering a pharmaceutically acceptable carrier to the patient.

41. The method of clause 40, wherein the pharmaceutically acceptable carrier is for parenteral administration or topical administration.

42. The method of any one of clauses 39 to 41, wherein the patient has a disease selected from the group consisting of cancer, a muscular disorder, hematological diseases or bone marrow failure states including myelodysplastic syndrome and severe aplastic anemia, a pulmonary disorder, a skin disorder, a neurological disease, neurofibromatosis 1, and a hemoglobinopathy.

43. The method of clause 42, wherein the cancer is selected from the group consisting of lung cancer, bone cancer, pancreatic cancer, skin cancer, uterine cancer, ovarian cancer, endometrial cancer, rectal cancer, stomach cancer, colon cancer, breast cancer, cancer of the esophagus, cancer of the endocrine system, prostate cancer, leukemia, lymphoma, mesothelioma, cancer of the bladder, cancer of the kidney, neoplasms of the central nervous system, brain cancer, and adenocarcinoma.

44. The method of clause 42, wherein the skin disorder is a Staphylococcus aureus infection.

45. The method of clause 42, wherein the muscular disorder is muscular dystrophy.

46. The method of any one of clauses 39 to 45, wherein the nucleic acid nanostructure delivery composition is not cytotoxic to the cells of the patient.

47. The method of any one of clauses 39 to 46, wherein the method comprises administering a first composition of any of clauses 1 to 38 or clause 68 and the method further comprises administering a second composition of any of clauses 1 to 31 or 33 to 38 or clause 68 comprising a different small molecule therapeutic than the first composition.

48. The method of any one of clauses 39 to 46, wherein the method further comprises administering a nucleic acid nanostructure delivery composition comprising one or more nucleic acid payloads, or another macromolecule selected from an antibody, a polypeptide, or an antibody-drug conjugate.

49. The method of clause 48, wherein the nucleic acid comprises DNA or RNA.

50. The method of clause 48 or 49, wherein the payload nucleic acid is used for homology directed repair or as transposable elements.

51. The method of any one of clauses 48 to 50, wherein the payload nucleic acids comprise a short guide RNA (sgRNA) or a donor DNA strand.

52. The method of clause 51, wherein the sgRNA is used for targeting an enzyme to a specific genomic sequence.

53. The method of any one of clauses 48 to 52, wherein the payload further comprises a CRISPR associated enzyme.

54. The method of clause 52, wherein the targeted enzyme is a CRISPR associated enzyme.

55. The method of any one of clauses 48 to 54, wherein the payload comprises a CRISPR associated enzyme, an sgRNA, or a donor DNA strand.

56. The method of any one of clauses 48 to 55, wherein the payload further comprises a Cas9 nuclease enzyme.

57. The method of any one of clauses 48 to 56, wherein the payloads comprise Cas9, Cas10, an sgRNA, or a donor DNA strand.

58. The method of any one of clauses 48 to 57, wherein the payload further comprises a Cas 9 enzyme that is deactivated (dCas9) and is fused with a deaminase.

59. The method of any one of clauses 48 to 58, wherein the payloads comprise a coding sequence for Cas9, an sgRNA, or a donor DNA strand in the form of a plasmid.

60. The method of any one of clauses 48 to 59, wherein the payloads consist of one molecule each of CRISPR/Cas9, an sgRNA, or a donor DNA strand.

61. The method of clause 48 or 49, wherein the payload comprises an antisense oligonucleotide.

62. The method of clause 48 or 49, wherein the payload is of a size selected from the group consisting of 0.1 kB or more, 0.2 kB or more, 0.3 kB or more, 0.4 kB or more, 0.5 kB or more, 0.6 kB or more, 0.7 kB or more, 0.8 kB or more, 0.9 kB or more, 1 kB or more, 1.5 kB or more, 2 kB or more, 2.5 kB or more, 3 kB or more, 3.5 kB or more, 4 kB or more, 4.5 kB or more, 5 kB or more, 5.5 kB or more, 6 kB or more, 6.5 kB or more, 7 kB or more, 7.5 kB or more, 8 kB or more, and 8.5 kB or more.

63. The method of any one of clauses 48 to 62, wherein the nucleic acid nanostructure delivery composition comprises one or more oligonucleotides with overhangs that bind through complementary base paring with the payload nucleic acids.

64. The method of any one of clauses 48 to 62, wherein the payload is associated with the nucleic acid nanostructure delivery composition by a high affinity, non-covalent bond interaction between a biotin molecule on the payload and a molecule that binds to biotin on the nucleic acid nanostructure delivery composition.

65. The method of clause 64, wherein molecule that binds to biotin is bound to the nucleic acid nanostructure delivery composition by a covalent phosphonamidite bond formed via an EDC-NHS coupling reaction between a terminal phosphate group of a 5′ end of an overhang on the nucleic acid nanostructure delivery composition and an amine group on the molecule that binds to biotin.

66. The method of any one of clauses 1 to 65, wherein the nucleic acid nanostructure delivery composition has an aspect ratio of about 2.

67. The method of any one of clauses 39 to 66 wherein the small molecule therapeutic and the nucleic acid nanostructure delivery composition both induce anti-tumor immunity.

68. The composition of any one of clauses 1 to 9 or 19 to 38, wherein the small molecule therapeutic is associated with the nucleic acid nanostructure delivery composition by base pairing wherein the small molecule therapeutic comprises a nucleic acid covalently bound to the small molecule therapeutic and wherein the nucleic acid covalently bound to the small molecule therapeutic is base-paired to a complementary nucleic acid on the nucleic acid nanostructure delivery composition.

69. The method of any one of clauses 39 to 43 or 46 to 67, wherein the small molecule therapeutic induces anti-tumor immunity by targeting cancer cells.

70. The method of any one of clauses 39 to 43 or 46 to 67, wherein the small molecule therapeutic induces anti-tumor immunity by targeting immunosuppressive cells.

71. The method of clause 70 wherein the immunosuppressive cells are selected from tumor-associated macrophages and myeloid-derived suppressor cells.

### BRIEF DESCRIPTION OF THE DRAWINGS

FIG. 1 shows the absorbance spectra of TLBST in Tris buffer pH 7.4 with a peak at 208 nm and (inset) concentration calibration curves for different time points.

FIGS. 2A-2C show that TLBST can be loaded onto DNAO cuboids without causing structural changes to the DNAO. FIG. 2A: 1% agarose-gel electrophoresis of TLBST loaded cuboids run at 70 volts, 2 hours in a 0.5×TBE solution with 11 mM MgCl₂. (i) 1 kb ladder; (ii) 7560-scaffold (iii) cuboids at 9 nM; cuboids at 9 nM incubated for 2 hours with TLBST at (iv) 1.25 mg/mL; (v) 0.625 mg/mL; and (vi) 0.3125 mg/mL dissolved in 40 mM Tris buffer with 10 mM MgCl₂. FIGS. 2B-2C: Negative stain TEM images of (FIG. 2B) unloaded DNAO cuboids and (inset) enlarged view of individual unloaded cuboids; (FIG. 2C) DNAO cuboids after 2 hr 0.625 mg/ml TLBST incubation and (inset) enlarged view of individual loaded cuboids; Scale bars are 100 nm and 20 nm (inset).

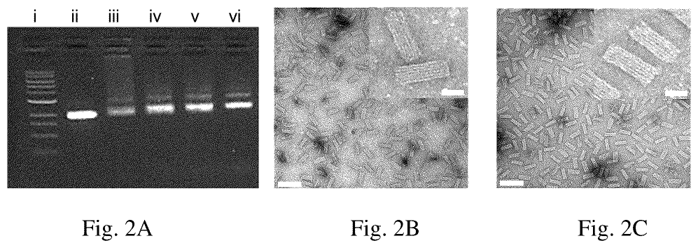

FIG. 3 shows a schematic of the methods of Example 3.

FIGS. 4A-4E show that DNAO-TLBST induces cytotoxicity and concomitant IL-1β, IL-18, and IFNβ release in murine macrophages. FIG. 4A: Viability measured as percentage MTT signal from cells treated as indicated relative to untreated controls. FIG. 4B: Cytotoxicity measured as percentage lactate dehydrogenase (LDH) release in the supernatant of RAW264.7 cells 24 hours after the indicated treatment relative to maximum LDH release from controls lysed at the time of sample collection. FIGS. 4C-4E: Concentrations of IL-1β, IL-18, and IFNβ in the supernatant 24 hours after the indicated treatment. N=3 replicate wells/group. Bars represent mean values with standard error of the mean (SEM). *P<0.05, **P<0.01, ***P<0.001, ****P<0.0001 by one-way ANOVA with Tukey post-test. In all panels of this figure, the left-most bar is media, the second bar from the left is a DNAO control, the third bar from the left is free TLBST, and the right-most bar is DNAO-TLBST.

FIG. 5 shows a schematic of Example 4 methodology.

FIGS. 6A-6D show that DNAO-TLBST induces cytotoxicity and concomitant IL-1β, IL-18, and IFNβ release in human macrophages. FIG. 6A: Cytotoxicity measured as percentage LDH release in the supernatant of macrophage-differentiated THP-1 cells 24 hours after the indicated treatment relative to maximum LDH release from controls lysed at the time of sample collection. FIGS. 6B-6D: Concentrations of IL-1β, IL-18, and IFNβ in supernatant 24 hours after the indicated treatment. N=3 replicate wells/group. Bars represent mean values with SEM. *P<0.05, **P<0.005, ***P<0.001, ****P<0.0001 by one-way ANOVA with Tukey post-test. In all panels of this figure, the left-most bar is media, the second bar from the left is a DNAO control, the third bar from the left is free TLBST, and the right-most bar is DNAO-TLBST.

FIG. 7 shows that DNAO-TLBST induces mild cytotoxicity in human prostate epithelial cells. Cytotoxicity measured as percentage LDH release in the supernatant of PC-3 cells 24 hours after the indicated treatment relative to maximum LDH release from controls lysed at the time of cell stimulation. N=3 replicate wells/group. Bars represent mean values with SEM. *P<0.05 by one-way ANOVA with Tukey post-test. In this figure, the left-most bar is media, the second bar from the left is a DNAO control, the third bar from the left is free TLBST, and the right-most bar is DNAO-TLBST.

### DETAILED DESCRIPTION OF ILLUSTRATIVE EMBODIMENTS

The invention relates to nucleic acid nanostructure delivery compositions for non-viral delivery of small molecule therapeutics, and methods therefor. More particularly, the invention relates to nucleic acid nanostructure delivery compositions, such as DNA origami compositions, for the delivery, for example, of small molecule therapeutics, and methods therefor.

The following clauses, and combinations thereof, provide various additional illustrative aspects of the invention described herein. The various embodiments described in any other section of this patent application, including the summary portion of the section titled “BACKGROUND AND SUMMARY”, the “EXAMPLES”, and this “DETAILED DESCRIPTION OF ILLUSTRATIVE EMBODIMENTS” section of the application are applicable to any of the following embodiments of the invention described in the numbered clauses below.

1. A composition comprising a non-viral delivery vehicle comprising a nucleic acid nanostructure delivery composition, and a small molecule therapeutic.

2. The composition of clause 1, wherein the nucleic acid nanostructure delivery composition comprises a DNA origami composition.

3. The composition of clause 1, wherein the nucleic acid nanostructure delivery composition comprises single-stranded or double-stranded DNA or RNA.

4. The composition of clause 3, wherein the nucleic acid nanostructure delivery composition comprises DNA.

5. The composition of clause 3, wherein the nucleic acid nanostructure delivery composition comprises RNA.

6. The composition of clause 3 wherein the nucleic acid nanostructure delivery composition comprises RNA and DNA.

7. The composition of any one of clauses 3 and 4 to 6, wherein the nucleic acid nanostructure delivery composition comprises both single-stranded and double-stranded regions of the nucleic acids.

8. The composition of any one of clauses 3 to 6, wherein the nucleic acid nanostructure delivery composition is single-stranded.

9. The composition of any one of clauses 3 to 6, wherein the nucleic acid nanostructure delivery composition is double-stranded.

10. The composition of any one of clauses 1 to 9, wherein the small molecule therapeutic is associated with the nucleic acid nanostructure delivery composition by a high affinity, non-covalent bond interaction between a biotin molecule on the small molecule therapeutic and a molecule that binds to biotin on the nucleic acid nanostructure delivery composition.

11. The composition of clause 10, wherein the molecule that binds to biotin is bound to the nucleic acid nanostructure delivery composition by a covalent phosphonamidite bond formed via an EDC-NHS coupling reaction between a terminal phosphate group of a 5′ end of an overhang on the nucleic acid nanostructure delivery composition and an amine group on the molecule that binds to biotin.

12. The composition of clause 10 or 11, wherein the biotin is bound to the small molecule therapeutic by a covalent bond.

13. The composition of any one of clauses 1 to 9, wherein the small molecule therapeutic is bound to the nucleic acid nanostructure delivery composition by a covalent bond.

14. The composition of clause 13, wherein the covalent bond is formed via an EDC-NHS coupling reaction between a terminal phosphate group of the 5′ end of an overhang on the nucleic acid nanostructure delivery composition and an amine group on the small molecule therapeutic.

15. The composition of clause 13, wherein the covalent bond is formed via a click chemistry coupling reaction between an azide group on the nucleic acid nanostructure delivery composition and an alkyne group on the small molecule therapeutic.

16. The composition of clause 13, wherein the covalent bond is formed via a click chemistry coupling reaction between an azide group on the small molecule therapeutic and an alkyne group on the nucleic acid nanostructure delivery composition.

17. The composition of any one of clauses 1 to 9, wherein the small molecule therapeutic is associated with the nucleic acid nanostructure delivery composition by a covalent bond between a carboxy terminated molecule on the nucleic acid nanostructure delivery composition and a primary amine on the small molecule therapeutic.

18. The composition of any one of clauses 1 to 9, wherein the small molecule therapeutic is associated with the nucleic acid nanostructure delivery composition by an electrostatic interaction between a negatively charged nucleic acid nanostructure delivery composition and a positively charged amine in the small molecule therapeutic.

19. The composition of any one of clauses 1 to 9, wherein the small molecule therapeutic is associated with the nucleic acid nanostructure delivery composition by intercalation of the small molecule therapeutic into the nucleic acid nanostructure delivery composition.

20. The composition of any one of clauses 1 to 19, wherein the small molecule therapeutic causes pyroptosis.

21. The composition of any one of clauses 1 to 19, wherein the small molecule therapeutic causes apoptosis.

22. The composition of any one of clauses 1 to 19, wherein the small molecule therapeutic causes necroptosis.

23. The composition of any one of clauses 1 to 18 and 20, wherein the small molecule therapeutic is a post-proline cleaving enzyme inhibitor.

24. The composition of clause 23, wherein the small molecule therapeutic inhibits a post-proline cleaving dipeptidyl peptidase (DPP) selected from DPP4, DPP8, DPP9, and fibroblast activation protein.

25. The composition of any one of clauses 1 to 24, wherein the small molecule therapeutic induces anti-tumor immunity.

26. The composition of any one of clauses 1 to 25, wherein the small molecule therapeutic induces cytokine production.

27. The composition of any one of clauses 1 to 26, wherein the small molecule therapeutic induces inflammasome activation.

28. The composition of clause 26, wherein the small molecule therapeutic induces the production of an interferon or an interleukin.

29. The composition of clause 28, wherein the interferon and the interleukin are selected from a type one interferon, IFN-β, IFN-γ, IL-1β, IL-6, IL-12p70, and IL-18.

30. The composition of clause 26, wherein the cytokine is selected from TNF-α and MCP-1/CCL2.

31. The composition of any one of clauses 1 to 30, wherein the small molecule therapeutic causes cancer cell lysis.

32. The composition of any one of clauses 1 to 18, 20, and 23 to 31, wherein the small molecule therapeutic is talabostat.

33. The composition of any one of clauses 1 to 32, wherein the nucleic acid nanostructure delivery vehicle comprises a cell-targeting molecule.

34. The composition of clause 33, wherein the cell-targeting molecule is selected from an antibody, an aptamer, a peptide, PNA, and a small molecule cell-targeting molecule.

35. The composition of clause 34, wherein the cell-targeting molecule is IL4Pep1.

36. The composition of any one of clauses 1 to 35, wherein the nucleic acid nanostructure delivery composition is coated with one or more polymers.

37. The composition of clause 36, wherein the one or more polymers comprise PEG-poly-L-lysine.

38. The composition of clause 32, wherein the talabostat modulates the activity of a molecule selected from DPP, NLRP1, CARD8, and a gasdermin family member.

39. A method of treating a patient with a disease, the method comprising administering to the patient any of the compositions of clauses 1 to 38 or clause 68, and treating the disease in the patient.

40. The method of clause 39, further comprising administering a pharmaceutically acceptable carrier to the patient.

41. The method of clause 40, wherein the pharmaceutically acceptable carrier is for parenteral administration or topical administration.

42. The method of any one of clauses 39 to 41, wherein the patient has a disease selected from the group consisting of cancer, a muscular disorder, hematological diseases or bone marrow failure states including myelodysplastic syndrome and severe aplastic anemia, a pulmonary disorder, a skin disorder, a neurological disease, neurofibromatosis 1, and a hemoglobinopathy.

43. The method of clause 42, wherein the cancer is selected from the group consisting of lung cancer, bone cancer, pancreatic cancer, skin cancer, uterine cancer, ovarian cancer, endometrial cancer, rectal cancer, stomach cancer, colon cancer, breast cancer, cancer of the esophagus, cancer of the endocrine system, prostate cancer, leukemia, lymphoma, mesothelioma, cancer of the bladder, cancer of the kidney, neoplasms of the central nervous system, brain cancer, and adenocarcinoma.

44. The method of clause 42, wherein the skin disorder is a Staphylococcus aureus infection.

45. The method of clause 42, wherein the muscular disorder is muscular dystrophy.

46. The method of any one of clauses 39 to 45, wherein the nucleic acid nanostructure delivery composition is not cytotoxic to the cells of the patient.

47. The method of any one of clauses 39 to 46, wherein the method comprises administering a first composition of any of clauses 1 to 38 or clause 68 and the method further comprises administering a second composition of any of clauses 1 to 31 or 33 to 38 or clause 68 comprising a different small molecule therapeutic than the first composition.

48. The method of any one of clauses 39 to 46, wherein the method further comprises administering a nucleic acid nanostructure delivery composition comprising one or more nucleic acid payloads, or another macromolecule selected from an antibody, a polypeptide, or an antibody-drug conjugate.

49. The method of clause 48, wherein the nucleic acid comprises DNA or RNA.

50. The method of clause 48 or 49, wherein the payload nucleic acid is used for homology directed repair or as transposable elements.

51. The method of any one of clauses 48 to 50, wherein the payload nucleic acids comprise a short guide RNA (sgRNA) or a donor DNA strand.

52. The method of clause 51, wherein the sgRNA is used for targeting an enzyme to a specific genomic sequence.

53. The method of any one of clauses 48 to 52, wherein the payload further comprises a CRISPR associated enzyme.

54. The method of clause 52, wherein the targeted enzyme is a CRISPR associated enzyme.

55. The method of any one of clauses 48 to 54, wherein the payload comprises a CRISPR associated enzyme, an sgRNA, or a donor DNA strand.

56. The method of any one of clauses 48 to 55, wherein the payload further comprises a Cas9 nuclease enzyme.

57. The method of any one of clauses 48 to 56, wherein the payloads comprise Cas9, Cas10, an sgRNA, or a donor DNA strand.

58. The method of any one of clauses 48 to 57, wherein the payload further comprises a Cas 9 enzyme that is deactivated (dCas9) and is fused with a deaminase.

59. The method of any one of clauses 48 to 58, wherein the payloads comprise a coding sequence for Cas9, an sgRNA, or a donor DNA strand in the form of a plasmid.

60. The method of any one of clauses 48 to 59, wherein the payloads consist of one molecule each of CRISPR/Cas9, an sgRNA, or a donor DNA strand.

61. The method of clause 48 or 49, wherein the payload comprises an antisense oligonucleotide.

62. The method of clause 48 or 49, wherein the payload is of a size selected from the group consisting of 0.1 kB or more, 0.2 kB or more, 0.3 kB or more, 0.4 kB or more, 0.5 kB or more, 0.6 kB or more, 0.7 kB or more, 0.8 kB or more, 0.9 kB or more, 1 kB or more, 1.5 kB or more, 2 kB or more, 2.5 kB or more, 3 kB or more, 3.5 kB or more, 4 kB or more, 4.5 kB or more, 5 kB or more, 5.5 kB or more, 6 kB or more, 6.5 kB or more, 7 kB or more, 7.5 kB or more, 8 kB or more, and 8.5 kB or more.

63. The method of any one of clauses 48 to 62, wherein the nucleic acid nanostructure delivery composition comprises one or more oligonucleotides with overhangs that bind through complementary base paring with the payload nucleic acids.

64. The method of any one of clauses 48 to 62, wherein the payload is associated with the nucleic acid nanostructure delivery composition by a high affinity, non-covalent bond interaction between a biotin molecule on the payload and a molecule that binds to biotin on the nucleic acid nanostructure delivery composition.

65. The method of clause 64, wherein molecule that binds to biotin is bound to the nucleic acid nanostructure delivery composition by a covalent phosphonamidite bond formed via an EDC-NHS coupling reaction between a terminal phosphate group of a 5′ end of an overhang on the nucleic acid nanostructure delivery composition and an amine group on the molecule that binds to biotin.

66. The method of any one of clauses 1 to 65, wherein the nucleic acid nanostructure delivery composition has an aspect ratio of about 2.

67. The method of any one of clauses 39 to 66 wherein the small molecule therapeutic and the nucleic acid nanostructure delivery composition both induce anti-tumor immunity.

68. The composition of any one of clauses 1 to 9 or 19 to 38, wherein the small molecule therapeutic is associated with the nucleic acid nanostructure delivery composition by base pairing wherein the small molecule therapeutic comprises a nucleic acid covalently bound to the small molecule therapeutic and wherein the nucleic acid covalently bound to the small molecule therapeutic is base-paired to a complementary nucleic acid on the nucleic acid nanostructure delivery composition.

69. The method of any one of clauses 39 to 43 or 46 to 67, wherein the small molecule therapeutic induces anti-tumor immunity by targeting cancer cells.

70. The method of any one of clauses 39 to 43 or 46 to 67, wherein the small molecule therapeutic induces anti-tumor immunity by targeting immunosuppressive cells.

71. The method of clause 70 wherein the immunosuppressive cells are selected from tumor-associated macrophages and myeloid-derived suppressor cells.

In various embodiments, the nucleic acid nanostructure delivery compositions described herein may comprise any non-viral composition for in vivo delivery of the payloads, such as small molecule therapeutics. By way of example, the nucleic acid nanostructure delivery compositions described herein may be selected from the group comprising synthetic virus-like particles, carbon nanotubes, emulsions, and any nucleic acid nanostructure delivery composition, such as DNA origami structures. DNA origami structures are described in U.S. Pat. No. 9,765,341, incorporated herein by reference. In any of the nucleic acid nanostructure delivery composition embodiments described herein, the nucleic acid nanostructure can comprise M13 bacteriophage DNA.

In these embodiments, the nucleic acid nanostructure delivery compositions have a high degree of tunability in structure and function, opportunities to protect payloads from adverse reactions or degradation by the immune system, and cell targeting via surface charge, particle size, or conjugation with various aptamers. These delivery systems also lend themselves to computer aided design, and they have suitable pathways to robust, commercial scale manufacturing processes with higher yields and fewer purification steps than viral manufacturing processes.

A nucleic acid nanostructure delivery composition (e.g., a DNA origami structure), as a delivery platform, is programmable and offers an opportunity for precise scale-up and manufacturing. In this embodiment, the biologic and non-viral nature of the nucleic acid nanostructure delivery composition reduces the chance of adverse immune reactions. In this embodiment, control of each nucleotide that forms a part of the nucleic acid nanostructure delivery composition (e.g., DNA origami nanostructure) allows for the precise design and modification of the structure, including suitable chemical moieties which can make in vivo delivery and endosomal escape possible. In various embodiments, the nucleic acid nanostructure delivery composition can comprise DNA or RNA. In various embodiments, the nucleic acid nanostructure delivery composition can be single-stranded or double-stranded or both, and can comprise DNA and RNA.

In this embodiment, the nucleic acid nanostructure delivery composition can undergo self-base pairing (i.e., a DNA origami structure) to fold into structures that can form the single-stranded or double-stranded scaffold that can encapsulate a payload, such as a small molecule therapeutic, or the scaffold can have both single-stranded and double-stranded regions.

In another illustrative embodiment, any of the nucleic acid nanostructure delivery compositions described herein can be coated with one or more polymers to protect the compositions from immune responses or to enhance endosomal escape. In one embodiment, the one or more polymers comprise cationic block co-polymers. In another embodiment, the one or more polymers comprise polyethylene glycol. In another embodiment, the one or more polymers comprise polyethylene glycol poly-L-lysine. In yet another embodiment, the one or more polymers comprise polyethylenimine. In an additional embodiment, the one or more polymers comprise polyethylene glycol poly-L-lysine and polyethylenimine.

In one embodiment, the small molecule therapeutic can be associated with the nucleic acid nanostructure delivery composition through, for example, a biotin-avidin interaction. In one aspect, a molecule that binds to biotin can be bound to the nucleic acid nanostructure delivery composition by a covalent phosphonamidite bond formed via an EDC-NHS coupling reaction between a terminal phosphate group of a 5′ end of an overhang on the nucleic acid nanostructure delivery composition and an amine group on the molecule that binds to biotin. In this embodiment, the biotin can be bound to the small molecule therapeutic by a covalent bond.

In another embodiment, the small molecule therapeutic can be bound to the nucleic acid nanostructure delivery composition by a covalent bond. In this embodiment, the covalent bond can be formed via an EDC-NHS coupling reaction between a terminal phosphate group of the 5′ end of an overhang on the nucleic acid nanostructure delivery composition and an amine group on the small molecule therapeutic. In another embodiment, the covalent bond can be formed via a click chemistry coupling reaction between an azide group on the nucleic acid nanostructure delivery composition and an alkyne group on the small molecule therapeutic. In yet another embodiment, the covalent bond can be formed via a click chemistry coupling reaction between an azide group on the small molecule therapeutic and an alkyne group on the nucleic acid nanostructure delivery composition. In still another aspect, the small molecule therapeutic can be associated with the nucleic acid nanostructure delivery composition by a covalent bond between a carboxy-terminated molecule on the nucleic acid nanostructure delivery composition and a primary amine on the small molecule therapeutic.

In another illustrative embodiment, the small molecule therapeutic can be associated with the nucleic acid nanostructure delivery composition by an electrostatic interaction between a negatively charged nucleic acid nanostructure delivery composition and a positively charged amine in the small molecule therapeutic. In still another embodiment, the small molecule therapeutic can be associated with the nucleic acid nanostructure delivery composition by intercalation of the small molecule therapeutic into the nucleic acid nanostructure delivery composition.

In yet another embodiment, the small molecule therapeutic can be associated with the nucleic acid nanostructure delivery composition by base pairing where the small molecule therapeutic comprises a nucleic acid covalently bound to the small molecule therapeutic and where the nucleic acid covalently bound to the small molecule therapeutic is base-paired to a complementary nucleic acid on the nucleic acid nanostructure delivery composition.

In various embodiments, the small molecule therapeutic can be any suitable small molecule therapeutic. Exemplary small molecule therapeutics include any small molecule therapeutic capable of modulating or otherwise modifying cell function, including pharmaceutically active compounds. Suitable molecules can include, but are not limited to, peptides, oligopeptides, retro-inverso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins, antigens and antibodies thereto, haptens and antibodies thereto, hormones, lipids, phospholipids, liposomes, toxins, antibiotics, analgesics, bronchodilators, beta-blockers, antimicrobial agents, antihypertensive agents, cardiovascular agents including antiarrhythmics, cardiac glycosides, antianginals and vasodilators, central nervous system agents including stimulants, psychotropics, antimanics, and depressants, antiviral agents, antihistamines, cancer drugs including chemotherapeutic agents, tranquilizers, anti-depressants, H-2 antagonists, anticonvulsants, antinauseants, prostaglandins and prostaglandin analogs, muscle relaxants, anti-inflammatory substances, stimulants, decongestants, antiemetics, diuretics, antispasmodics, antiasthmatics, anti-Parkinson agents, mineral and nutritional additives, and immunomodulatory agents.

Further, the small molecule therapeutic can be any drug known in the art which is cytotoxic, enhances tumor permeability, inhibits tumor cell proliferation, induces apoptosis, induces pyroptosis, induces necroptosis, is used to treat diseases caused by infectious agents, or enhances an endogenous immune response directed to cancer cells, such as by inhibiting immunosuppressive cells such as TAMs or MDSCs. Small molecule therapeutics suitable for use in accordance with this invention include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, paclitaxel, paclitaxel derivatives, Taxotere™., cyclophosphamide, daunomycin, rhizoxin, T2 toxin, plant alkaloids, prednisone, hydroxyurea, teniposide, mitomycins, discodermolides, microtubule inhibitors, epothilones, tubulysin, cyclopropyl benz[e]indolone, seco-cyclopropyl benz[e]indolone, 0-Ac-seco-cyclopropyl benz[e]indolone, bleomycin and any other antibiotic, nitrogen mustards, nitrosureas, vincristine, vinblastine, and analogs and derivative thereof such as deacetylvinblastine monohydrazide, colchicine, colchicine derivatives, allocolchicine, thiocolchicine, trityl cysteine, Halicondrin B, dolastatins such as dolastatin 10, amanitins such as alpha.-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, TLR agonists such as TLR7 or TLR9 agonists, PI3 kinase inhibitors, microtubule inhibitors, and any other art-recognized small molecule therapeutic.

In another embodiment, the small molecule therapeutic can be a tyrosine kinase inhibitor selected from the group consisting of Crizotinib, Ceritinib, Alectinib, Brigatinib, Lorlatinib, Capmatinib, Tepotinib, Gefitinib, Erlotinib, Lapatinib, Icotinib, Afatinib, Osimertinib, Neratinib, Dacomitinib, Almonertinib, Tucatinib, Midostaurin, Gilteritinib, Quizartinib, Pexidartinib, Sorafenib, Sunitinib, Pazopanib, Vandetanib, Axitinib, Cabozantinib, Regorafenib, Apatinib, Lenvatinib, Tivozanib, Fruquintinib, Nintedanib, Anlotinib, Erdafitinib, Pemigatinib, Avapritinib, Ripretinib, Selpercatinib, Pralsetinib, Larotrectinib, and Larotrectinib.

In another illustrative embodiment, the small molecule therapeutic can be a non-receptor tyrosine kinase inhibitor selected from the group consisting of Imatinib, Dasatinib, Nilotinib, Bosutinib, Radotinib, Ponatinib, Ibrutinib, Acalabrutinib, Zanubrutinib, Ruxolitinib, and Fedratinib.

In another aspect, the small molecule therapeutic can be a small molecule serine/threonine kinase inhibitor selected from the group consisting of Vemurafenib, Dabrafenib, Encorafenib, Trametinib, Cobimetinib, Binimetinib, Selumetinib, Palbociclib, Ribociclib, Abemaciclib, Idelalisib, Copanlisib, Duvelisib, Alpelisib, Temsirolimus, Everolimus, and Sirolimus. In another embodiment, the small molecule therapeutic can be an epigenetic target selected from the group consisting of Tazemetostat, Vorinostat, Romidepsin, Belinostat, Tucidinostat, Panobinostat, Enasidenib, and Ivosidenib. In still another aspect, the small molecule therapeutic can be a small molecule inhibitor of BCL-2, the hedgehog pathway, proteasome, or PARP selected from the group consisting of Venetoclax, Vismodegib, Sonidegib, Glasdegib, Bortezomib, Carfilzomib, Ixazomib, Olaparib, Rucaparib, Niraparib, and Talazoparib.

In another embodiment, the small molecule therapeutic can be a post-proline cleaving enzyme inhibitor. In one aspect, the small molecule therapeutic inhibits a post-proline cleaving dipeptidyl peptidase (DPP) selected from DPP4, DPP8, DPP9, and fibroblast activation protein. In this embodiment, the DPP inhibitors can be selected from Talabostat, Sitagliptin, Vildagliptin, Alogliptin, Saxagliptin, PSN-9301, R1438, TA-6666, PHX1149, GRC 8200, SYR-619, TS-021, SSR 162369, and ALS 2-0426. In one embodiment, the small molecule therapeutic can be the immunomodulatory agent talabostat. In another embodiment, the talabostat or another small molecule therapeutic can modulate the activity of a molecule selected from DPP, NLRP1, CARD8, and a gasdermin family member.

In yet other embodiments, the small molecule therapeutic can induce anti-tumor immunity, can induce inflammasome activation, can inactivate TAMs or MDSCs, may induce cancer cell lysis, and/or can induce the production of cytokines (e.g., an interferon or an interleukin). In another embodiment, the small molecule therapeutic can induce the production of an interferon and/or an interleukin selected from a type one interferon, IFN-β, IFN-γ, IL-1β, IL-6, IL-12p70, and IL-18. In another aspect, the small molecule therapeutic can induce the production of a cytokine selected from TNF-α and MCP-1/CCL2.

In yet another aspect, the nucleic acid nanostructure delivery vehicle can comprises a cell-targeting molecule. In one illustrative embodiment, the cell-targeting molecule is selected from an antibody, an aptamer, a peptide, PNA, and a small molecule cancer cell-targeting molecule. In other embodiments, the cell-targeting molecule can be a vitamin (e.g., folate), peptide ligands identified from library screens, tumor cell-specific peptides, tumor cell-specific aptamers, tumor cell-specific monoclonal or polyclonal antibodies, Fab or scFv (i.e., a single chain variable region) fragments of antibodies, small organic molecules derived from combinatorial libraries, growth factors, such as EGF, FGF, insulin, and insulin-like growth factors, and homologous polypeptides, somatostatin and its analogs, transferrin, steroid hormones, retinoids, various Galectins, delta-opioid receptor ligands, cholecystokinin A receptor ligands, ligands specific for angiotensin AT1 or AT2 receptors, and other molecules that bind specifically to a receptor preferentially expressed on the surface of cells, such as cancer cells. In one embodiment, the cell-targeting molecule is IL4Pep1.

In another aspect, a cell-targeting component can be a nucleotide that is an RNA that forms a ‘stem-and-loop’ structure. In this aspect, the nucleic acid nanostructure delivery composition can be designed so that the polynucleotide strands fold into three-dimensional structures via a series of highly tuned ‘stem-and-loop’ configurations. In this embodiment, the nucleic acid nanostructure delivery composition can have a high affinity for protein receptors expressed on specific cells resulting in targeting of the nucleic acid nanostructure delivery composition and the payload to the specific cells. In this embodiment, the polynucleotide that binds to the target cell receptor can bind in conjunction with a peptide aptamer. In another aspect, the nucleic acid nanostructure delivery composition can be folded so that, in the presence of certain biomarkers such as cell receptors, microRNA, DNA, RNA or an antigen, the self-base pairs are disrupted and the nucleic acid nanostructure delivery composition can unfold, resulting in the triggered release of the payload only in the presence of the specific biomarker. For example, a lock-and-key mechanism for triggered opening of a nucleic acid nanostructure delivery composition (e.g., a DNA origami construct) has been demonstrated previously (Andersen, et al., Nature, Vol. 459, pages 73-76(2009), incorporated by reference herein). In these embodiments, the use of the nucleic acid nanostructure delivery composition to create three-dimensional structures that target cells and tissues allows for more efficient delivery of payloads with fewer side effects, since the nucleic acid nanostructure delivery composition can have low immunogenicity, and the payload will be released only in the presence of RNA or peptide biomarkers, for example, that exist in the cytosol of target cells and tissues.

In another embodiment, a method of treating a patient with a disease is provided. The method comprises administering to the patient any of the nucleic acid nanostructure delivery compositions comprising a small molecule therapeutic described herein, and treating the disease in the patient. In this embodiment, the method can further comprise administering a pharmaceutically acceptable carrier to the patient.

In various embodiments, any suitable route for administration of the nucleic acid nanostructure delivery compositions associated with a small molecule therapeutic can be used including parenteral administration. Suitable routes for such parenteral administration include intravenous, intraarterial, intraperitoneal, intrathecal, epidural, intracerebroventricular, intraurethral, intrasternal, intracranial, intratumoral, intraosseous, intramuscular and subcutaneous delivery. In one embodiment, means for parenteral administration include needle (including microneedle) injectors, needle-free injectors and infusion techniques. In other embodiments, oral, pulmonary, or topical routes of administration can be used.

In one embodiment, the nucleic acid nanostructure delivery compositions with the small molecule therapeutic described herein may be formulated as pharmaceutical compositions for parenteral or topical administration. Such pharmaceutical compositions and processes for making the same are known in the art for both humans and non-human mammals. See, e.g., REMINGTON: THE SCIENCE AND PRACTICE OF PHARMACY, (1995) A. Gennaro, et al., eds., 19^{th} ed., Mack Publishing Co. Additional active ingredients may be included in the compositions.

In one illustrative aspect, parenteral formulations are typically aqueous solutions which may contain carriers or excipients such as salts, carbohydrates and buffering agents (preferably at a pH of from 3 to 9), but they may be more suitably formulated as a sterile non-aqueous solution or as a dried form to be used in conjunction with a suitable vehicle such as sterile, pyrogen-free water or sterile saline. The preparation under sterile conditions, by lyophilization to produce a sterile lyophilized powder for a parenteral formulation, may readily be accomplished using standard pharmaceutical techniques well-known to those skilled in the art. In one embodiment, the solubility of the composition used in the preparation of a parenteral formulation may be increased by the use of appropriate formulation techniques, such as the incorporation of solubility-enhancing agents.

In one illustrative embodiment, pharmaceutical compositions for parenteral administration comprise: a) a pharmaceutically active amount of the nucleic acid nanostructure delivery composition; b) a pharmaceutically acceptable pH buffering agent to provide a pH in the range of about pH 4.5 to about pH 9; c) an ionic strength modifying agent in the concentration range of about 0 to about 300 millimolar; and d) water soluble viscosity modifying agent in the concentration range of about 0.25% to about 10% total formula weight or any combinations of a), b), c) and d) are provided.

In various illustrative embodiments, the pH buffering agents for use in the compositions and methods described herein are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and nonionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. Typically, non-acidic viscosity enhancing agents, such as a neutral or a basic agent are employed in order to facilitate achieving the desired pH of the formulation.

In one embodiment, the solubility of the compositions described herein used in the preparation of a parenteral formulation may be increased by the use of appropriate formulation techniques, such as the incorporation of solubility-enhancing agents.

In other embodiments, the compositions described herein may be administered topically. A variety of dose forms and bases can be applied to the topical preparations, such as an ointment, cream, gel, gel ointment, plaster (e.g. cataplasm, poultice), solution, powders, and the like. These preparations may be prepared by any conventional method with conventional pharmaceutically acceptable carriers or diluents as described below.

For example, vaseline, higher alcohols, beeswax, vegetable oils, polyethylene glycol, etc. can be used. In the preparation of a cream formulation, fats and oils, waxes, higher fatty acids, higher alcohols, fatty acid esters, purified water, emulsifying agents etc. can be used. In the preparation of gel formulations, conventional gelling materials such as polyacrylates (e.g. sodium polyacrylate), hydroxypropyl cellulose, hydroxypropyl methyl cellulose, polyvinyl alcohol, polyvinylpyrrolidone, purified water, lower alcohols, polyhydric alcohols, polyethylene glycol, and the like are used. In the preparation of a gel ointment, an emulsifying agent (preferably nonionic surfactants), an oily substance (e.g. liquid paraffin, triglycerides, and the like), etc. are used in addition to the gelling materials as mentioned above. A plaster such as cataplasm or poultice can be prepared by spreading a gel preparation as mentioned above onto a support (e.g. fabrics, non-woven fabrics). In addition to the above-mentioned ingredients, paraffins, squalane, lanolin, cholesterol esters, higher fatty acid esters, and the like may optionally be used. Moreover, antioxidants such as BHA, BHT, propyl gallate, pyrogallol, tocopherol, etc. may also be incorporated. In addition to the above-mentioned preparations and components, there may optionally be used any other conventional formulations for incorporation with any other additives.

In various embodiments, the dosage of the nucleic acid nanostructure delivery composition can vary significantly depending on the patient condition, or the disease state being treated, the route of administration and tissue distribution, and the possibility of co-usage of other therapeutic treatments. The effective amount to be administered to a patient is based on body surface area, patient weight or mass, and physician assessment of patient condition. In various embodiments, the nucleic acid nanostructure delivery composition can be administered to a patient with a disease or a disorder selected from the group consisting of diabetes, cancer, a muscular disorder, hematological diseases or bone marrow failure states including myelodysplastic syndrome and severe aplastic anemia, a pulmonary disorder, a skin disorder, a neurological disease, neurofibromatosis 1 (NF1), and a hemoglobinopathy. In one embodiment, the cancer is selected from the group consisting of lung cancer, bone cancer, pancreatic cancer, skin cancer, uterine cancer, ovarian cancer, endometrial cancer, rectal cancer, stomach cancer, colon cancer, breast cancer, cancer of the esophagus, cancer of the endocrine system, prostate cancer, leukemia, lymphoma, mesothelioma, cancer of the bladder, cancer of the kidney, neoplasms of the central nervous system, brain cancer, and adenocarcinoma. In another embodiment, the skin disorder is a Staphylococcus aureus infection. In yet another embodiment, the muscular disorder is muscular dystrophy (e.g., Duchenne Muscular Dystrophy). In still another embodiment, the nucleic acid nanostructure delivery compositions are not cytotoxic to the cells of the patient.

In still another embodiment, the method of treatment described above can comprise administering a first composition of any of the nucleic acid nanostructure delivery compositions with small molecule therapeutics described herein, and the method can further comprise administering a second composition comprising a different small molecule therapeutic than the first composition, or a macromolecule such as a monoclonal antibody, a polypeptide, or an antibody-drug conjugate.

In yet another embodiment, the method of treatment described above can further comprise administering a nucleic acid nanostructure delivery composition comprising one or more nucleic acid payloads. In this embodiment, the nucleic acid nanostructure delivery composition can comprise overhangs that bind through complementary base paring with the payload nucleic acids.

As used herein, the term “complementary base pairing” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

In one aspect, the nucleic acid nanostructure delivery composition comprising a nucleic acid can encapsulate a nucleic acid of 3 kB or more or another genetic payload for delivery to target cells. In these embodiments, the nucleic acid can have a size of 3 kB or more and can be DNA or RNA. In other embodiments, the nucleic acid can have a size of about 0.1 kB or more, about 0.2 kB or more, about 0.3 kB or more, about 0.4 kB or more, about 0.5 kB or more, about 0.6 kB or more, about 0.7 kB or more, about 0.8 kB or more, about 0.9 kB or more, about 1 kB or more, about 1.5 kB or more, about 2 kB or more, about 2.5 kB or more, about 3 kB or more, about 3.1 kB or more, about 3.2 kB or more, about 3.3 kB or more, about 3.4 kB or more, about 3.5 kB or more, about 3.6 kB or more, about 3.7 kB or more, about 3.8 kB or more, about 3.9 kB or more, about 4 kB or more, about 4.1 kB or more, about 4.2 kB or more, about 4.3 kB or more, about 4.4 kB or more, about 4.5 kB or more, about 4.6 kB or more, about 4.7 kB or more, about 4.8 kB or more, about 4.9 kB or more, about 5 kB or more, about 5.1 kB or more, about 5.2 kB or more, about 5.3 kB or more, about 5.4 kB or more, about 5.5 kB or more, about 5.6 kB or more, about 5.7 kB or more, about 5.8 kB or more, about 5.9 kB or more, about 6 kB or more, about 6.1 kB or more, about 6.2 kB or more, about 6.3 kB or more, about 6.4 kB or more, about 6.5 kB or more, about 6.6 kB or more, about 6.7 kB or more, about 6.8 kB or more, about 6.9 kB or more, about 7 kB or more, about 7.1 kB or more, about 7.2 kB or more, about 7.3 kB or more, about 7.4 kB or more, about 7.5 kB or more, about 7.6 kB or more, about 7.7 kB or more, about 7.8 kB or more, about 7.9 kB or more, about 8 kB or more, about 8.1 kB or more, about 8.2 kB or more, about 8.3 kB or more, about 8.4 kB or more, and about 8.5 kB or more.

In the embodiment where a nucleic acid nanostructure delivery composition is used, computer aided design tools can predict the nucleotide sequence necessary to produce highly engineered nucleic acid nanostructure delivery compositions. For gene delivery, these nucleic acid nanostructure delivery compositions offer the advantages of encapsulation efficiency, as the size and shape of the structure can be tailored to fit the cargo. In another aspect, loading efficiency can be increased by incorporating nucleic acid payloads into the encapsulating nucleic acid nanostructure delivery composition itself.

In other embodiments, the nucleic acid payload can be associated with the nucleic acid nanostructure delivery composition by a high affinity, non-covalent bond interaction between a biotin molecule on the 5′ and/or the 3′ end of the nucleic acid payload and a molecule that binds to biotin on the nucleic acid nanostructure delivery composition. In this embodiment, the molecule that binds to biotin can be bound to the nucleic acid nanostructure delivery composition by a covalent phosphonamidite bond formed via an EDC-NHS coupling reaction between a terminal phosphate group of a 5′ end of an overhang on the nucleic acid nanostructure delivery composition and an amine group on the molecule that binds to biotin. In this embodiment, the biotin can be bound to the nucleic acid payload by a covalent bond.

In another illustrative embodiment, the nucleic acid payload can be bound to the nucleic acid nanostructure delivery composition by a covalent bond. In this embodiment, the covalent bond can be formed via an EDC-NHS coupling reaction between a terminal phosphate group of the 5′ end of an overhang on the nucleic acid nanostructure delivery composition and an amine group on an amino terminal nucleotide of the nucleic acid payload. In another embodiment, the covalent bond can be formed via a click chemistry coupling reaction between an azide group on the nucleic acid nanostructure delivery composition and an alkyne group on the nucleic acid payload. In yet another embodiment, the covalent bond can be formed via a click chemistry coupling reaction between an azide group on the nucleic acid payload and an alkyne group on the nucleic acid nanostructure delivery composition. In still another embodiment, the nucleic acid payload can be associated with the nucleic acid nanostructure delivery composition by a covalent bond between a carboxy terminated molecule on the nucleic acid nanostructure delivery composition and a primary amine on the nucleic acid payload at the 5′ and/or the 3′ end.

Illustrative nucleic acid payloads for the nucleic acid nanostructure delivery compositions described herein can include any one or a combination of compositions selected from the group comprising nucleic acids (e.g., DNA or RNA), pDNA, oligodeoxyribonucleic acids (ODNs), dsDNA, ssDNA, antisense oligonucleotides, antisense RNA, siRNA, messenger RNA, guide RNA (e.g., small guide RNA), ribonucleoproteins, donor DNA strands used in the CRISPR/Cas9 system, and enzymes can also be delivered, such as CRISPR-associated enzymes, e.g., Cas9, Cas10, other Cas enzymes, enzymes used in other gene editing systems, such as ZFNs, custom designed homing endonucleases, TALENS systems, other gene editing endonucleases, and reverse transcriptase.

Other illustrative payloads include DNA constructs such as chimeric antigen receptor (CAR) constructs. CAR-T cells are T cells expressing chimeric antigen receptors (CARs). The CAR is a genetically engineered receptor that is designed to target a specific antigen, for example, a tumor antigen. This targeting can result in cytotoxicity against the tumor, for example, such that CAR-T cells expressing CARs can target and kill tumors via the specific tumor antigens. CARs can comprise a recognition region, e.g., a single chain fragment variable (scFv) region derived from an antibody for recognition and binding to the antigen expressed by the tumor, an activation signaling domain, e.g., the CD3(chain of T cells can serve as a T cell activation signal in CARs, and a co-stimulation domain (e.g., CD137, CD28 or CD134) to achieve prolonged activation of T cells in vivo. In some aspects, CARs are large DNA constructs.

In another embodiment, the nucleic acid payload can be a nucleic acid (e.g., DNA or RNA) with a size selected from the group consisting of 0.1 kB or more, 0.2 kB or more, 0.3 kB or more, 0.4 kB or more, 0.5 kB or more, 0.6 kB or more, 0.7 kB or more, 0.8 kB or more, 0.9 kB or more, 1 kB or more, 1.5 kB or more, 2 kB or more, 2.5 kB or more, 3 kB or more, 3.1 kB or more, 3.2 kB or more, 3.3 kB or more, 3.4 kB or more, 3.5 kB or more, 3.6 kB or more, 3.7 kB or more, 3.8 kB or more, 3.9 kB or more, 4 kB or more, 4.1 kB or more, 4.2 kB or more, 4.3 kB or more, 4.4 kB or more, 4.5 kB or more, 4.6 kB or more, 4.7 kB or more, 4.8 kB or more, 4.9 kB or more, 5 kB or more, 5.1 kB or more, 5.2 kB or more, 5.3 kB or more, 5.4 kB or more, 5.5 kB or more, 5.6 kB or more, 5.7 kB or more, 5.8 kB or more, 5.9 kB or more, 6 kB or more, 6.1 kB or more, 6.2 kB or more, 6.3 kB or more, 6.4 kB or more, 6.5 kB or more, 6.6 kB or more, 6.7 kB or more, 6.8 kB or more, 6.9 kB or more, 7 kB or more, 7.1 kB or more, 7.2 kB or more, 7.3 kB or more, 7.4 kB or more, 7.5 kB or more, 7.6 kB or more, 7.7 kB or more, 7.8 kB or more, 7.9 kB or more, 8 kB or more, 8.1 kB or more, 8.2 kB or more, 8.3 kB or more, 8.4 kB or more, and 8.5 kB or more.

In various embodiments, the payload can be any one or more of the components of the CRISPR RNP system including a CRISPR-associated enzyme (e.g., Cas9), a short guide RNA (sgRNA), and a donor DNA strand. In an embodiment where the payload comprises Cas9, Cas9 can be fused to a deaminase. In yet another embodiment, the nucleic acid payload can comprise an sgRNA used for targeting an enzyme to a specific genomic sequence. In another aspect, the targeted enzyme can be a CRISPR-associated enzyme. In another illustrative aspect, the payload can comprise one molecule each of CRISPR/Cas9, an sgRNA, and a donor DNA strand in the nucleic acid nanostructure delivery compositions described herein. In another embodiment, the payloads can be nucleic acids used for homology directed repair or as transposable elements. In yet another embodiment, the payloads can be any of the payloads described herein in the form of a plasmid construct.

In one aspect, the nucleic acid nanostructure delivery composition described herein can encapsulate a payload that is used for gene editing. In one aspect, the CRISPR/Cas9 system can be the payload and can be used for gene editing. In another embodiment, another gene editing system can be the payload, such as ZFNs, custom designed homing endonucleases, and TALENS systems. In the embodiment where the CRISPR/Cas9 system is the payload, the Cas9 endonuclease is capable of introducing a double strand break into a DNA target sequence. In this aspect, the Cas9 endonuclease is guided by the guide polynucleotide (e.g., sgRNA) to recognize and optionally introduce a double strand break at a specific target site into the genome of a cell. In this illustrative embodiment, the Cas9 endonuclease can unwind the DNA duplex in close proximity to the genomic target site and can cleave both target DNA strands upon recognition of a target sequence by a guide polynucleotide (e.g., sgRNA), but only if the correct protospacer-adjacent motif (PAM) is approximately oriented at the 3′ end of the target. In this embodiment, the donor DNA strand can then be incorporated into the genomic target site. The CRISPR/Cas9 system for gene editing is well-known in the art.

In another illustrative embodiment, the payload may include DNA segments that serve as nuclear localization signals, enhancing nuclear delivery of the nucleic acid nanostructure delivery compositions upon endosomal escape. In another aspect, the nucleic acid payload may include a nucleotide sequence designed to bind as an aptamer to endosomal receptors, enhancing intracellular trafficking of the nucleic acid nanostructure delivery compositions.

In one illustrative aspect, a nucleic acid nanostructure delivery composition (e.g., DNA origami) is provided to package the Cas9 protein, the sgRNA and the single stranded donor DNA strand together in one nanostructure to ensure co-delivery of all the components to a particular location at the same time. In this embodiment, the single stranded nature of the sgRNA and the donor DNA strand can be used to convert these components into constitutive parts of the nucleic acid nanostructure delivery composition (e.g., the DNA origami structure) such that they get delivered together and dissociate at the same time from the DNA nanostructure delivery composition upon reaching the target site (e.g., a target cell). In this embodiment, the DNA nanostructure delivery composition can deliver either a plasmid or the ribonucleoprotein (RNP) form of CRISPR/Cas 9.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

While the concepts of the present disclosure are susceptible to various modifications and alternative forms, specific embodiments thereof have been shown by way of example in the appended drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure.

### Example 1

### Self-Assembly and Characterization of DNA Origami Structures

The DNAO nanostructures were designed using CaDNAno software and were self-assembled by folding a 7560-nucleotide long M13mp18 single stranded scaffold and 22-58 nucleotide long single stranded oligonucleotides staples. The scaffold and staples were mixed at a 1:2 ratio in a solution containing 10 mM EDTA. 50 mM TrisBase, 50 mM NaCl, 200 mM MgCl₂, and DI water. The mixture was then allowed to undergo a 42 hour thermal annealing process where it was heated to 65° C. for 1 hour and then cooled at a rate of 1° C. after which it was held at a temperature of 4° C. till needed. A polyethylene glycol (PEG) based precipitation method was used to purify the excess staple strands after folding. For this, a target volume of DNAO nanostructure was mixed with an equal volume of 15% PEG8000 and centrifuged at 16,000 g for 30 minutes. The supernatant was removed and DNAO nanostructures were resuspended in a Tris EDTA buffer with 20 mM MgCl₂

### Example 2

Functionalize, Load, and Characterize Cell-Targeting Dnao-Tlbst Nanostructures

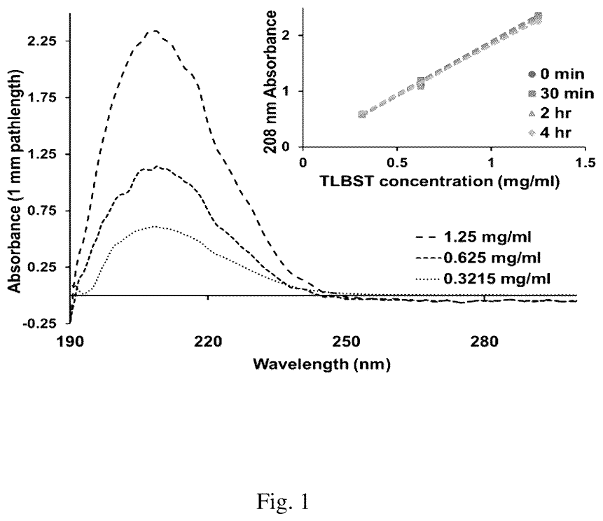

DNA origami (DNAO) can prevent drug degradation via encapsulation and decrease the minimum effective dose by selective delivery of drugs to target tissues. Thus, talabostat (TLBST)-loaded DNAO nanocarriers were designed and synthesized. First, a uv-vis spectroscopy-based method for quantifying TLBST concentration in solution was developed. Unlike Doxirubicin (Dox), which is well-characterized for nanoparticle based clinical studies, TLBST has only been delivered in its free form. Also, unlike Dox, which is a red powder with a florescence spectrum in the visible range, Talabostat mesylate (MW=310.18 Da) is a colorless powder with no absorption in the visible spectra. It was determined that TLBST has a stable absorption in the deep-uv region at ˜208 nm (FIG. 1). Calibration curves created at 4 time points using the peak absorbance values at 208 nm, FIG. 1 (inset), had R²>0.98 indicating a linear and stable correlation.

A solid cuboid shape with an aspect ratio of 2.3 (50 nm×21 nm×16 nm) was chosen for the nanocarriers as it is correlated with increased cellular uptake in vitro. Electrostatic TLBST loading was carried out at different concentrations and durations. We have previously functionalized cuboid DNAO with biotin and fluorophores, stabilized them against nuclease degradation using polyethylene glycol (PEG)-Poly-L-Lysine (PEG-PLL), and demonstrated cellular uptake of Cy5 functionalized and PEG-PLL DNAO in HEK 293T cells (data not shown).

### Example 3

Optimization of Cuboid DNAO Functionalization with a Cell-targeting Peptide, Confirmation of Specificity of Uptake in Vitro, and Characterization of Drug Loading Efficiency and Structural Stability in the Presence of Serum Nucleases

DNAO nanostructures can be functionalized in multiple ways. In some cases, molecules of interest are conjugated to an oligonucleotide which is then hybridized to a complementary region on an extended staple strand (handle or overhang) on the DNAO structure. The cell-targeting peptide (CTP) will be conjugated to a charge neutral peptide nucleic acid, PNA, oligonucleotide instead of a DNA oligonucleotide. PNAs are synthetic polymers of repeating peptide-like amide units (N-(2-aminoethyl) glycine) that mimic nucleic acids in their hybridization affinity and specificity via base-pairing and are becoming a widely used research tool in therapeutics. Their uncharged backbones lead to higher binding affinity with DNA than DNA:DNA which makes them attractive for binding to proteins and peptides. We will systematically optimize the conjugation of IL4RPep1 to PNA and DNA oligonucleotides and compare functionalization yields of the two. PNA-IL4R-pep1 and DNA-IL4R-pep1 conjugates will be synthesized by coupling azide-modified PNA and DNA oligonucleotides with alkyne modified IL4R-pep1 via click chemistry. Cuboid DNAO will be designed using CaDNAno and self-assembled by folding a 7560-nucleotide long M13 scaffold (tilibit nanosystems) and staple oligonucleotides (Integrated DNA Technologies) using published protocols (Wagenbauer, K. F. et al. How we make DNA origami. ChemBioChem 18, 1873-1885 (2017). Staple strands for fluorophore and CTP functionalization will be included as needed. The quality and robustness of the PEG purified structures will be assessed by using both agarose gel electrophoresis (AGE) and TEM. The assembly of CTP-DNAO will be optimized by hybridizing the PNA-CTP and DNA-CTP to their complementary overhangs on the DNAO by incubating at molar excesses of the DNA/PNA-CTP (2×-10×) and for different durations (30 min-2 hr) at 35° C. Excess DNA/PNA-CTP will be removed using ultracentrifugation. Product yield and degree of conjugation will be characterized using uv-vis spectroscopy.

The effect of CTP functionalization on DNAO uptake will be determined in THP-1 cells. Features of pyroptosis were first observed in macrophages treated with anthrax lethal toxin or infected with Shigella flexneri or Samonella, and macrophages remain the most well-utilized cell type in pyroptosis research. As such, we will induce macrophage differentiation of THP-1 cells with phorbol myristate acetate (PMA). Macrophage-differentiated THP-1 cells exhibit both cytotoxicity and IL-1β release in response to free TLBST, making them ideal for our cellular analyses. We will target DNAO nanocarriers to THP-1 cells using the IL4R-pep1 CTP peptide, which is used to localize therapeutics to both mouse and human tumor cells and tumor-associated macrophages. Each DNAO cuboid will be tagged with the same number of Cy5 fluorophores for visualizing cargo delivery in vitro. Differentiated THP-1 cells will be seeded in 24-well-plates and cultured overnight. The cells will then be incubated with either buffer as no treatment, bare DNAO, or DNAO-CTP for 12-24 h. After incubation, the cells will be washed, and fluorescence will be measured flow cytometrically.

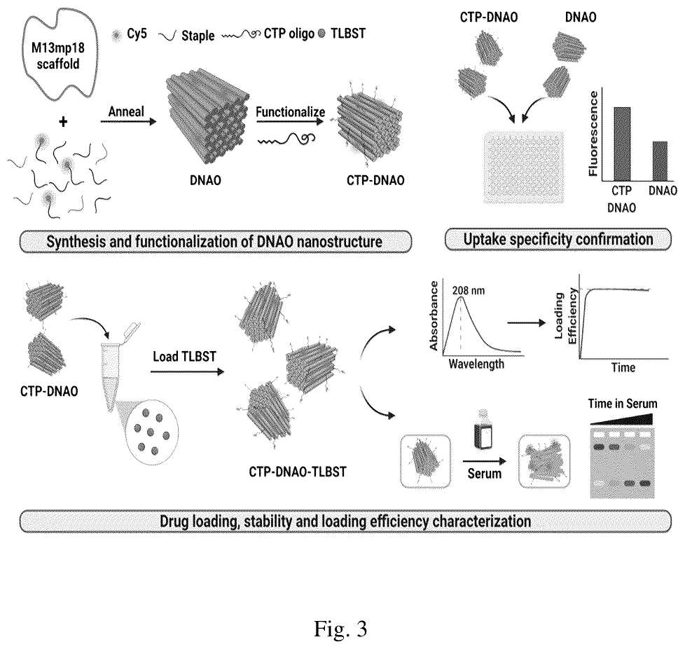

Although we have demonstrated a method to electrostatically load TLBST onto DNAO, we have not characterized TLBST loading efficiency (LE) across a range of DNAO concentrations or for TLBST concentrations <0.3125 mg/ml. To load TLBST, DNAO (final concentrations of 10 nM-30 nM) will be mixed with TLBST (final concentrations of 0.08-1.25 mg/ml) and shaken on a stir plate continuously for 0.5-4 hours. The mixture will be filtered via ultracentrifugation and concentration of the excess TLBST will be measured using uv-vis. LE will be calculated. Finally, the stability of these structures will be tested in the presence of nucleases at 37° C. Cell culture media supplemented with mammalian serum (fetal bovine serum (FBS)), which contains nucleases, will be used as a substitute for physiological in vivo conditions. DNAO will be incubated in RPMI 1640 culture medium containing 10% FBS for 2-24 hr. The incubated products will be analyzed with AGE to quantify degradation due to serum nucleases. See FIG. 3 for a schematic of the methods of this example.

### Example 4

DNAO-TLBST Induces Cytotoxicity and Concomitant IL-1β, IL-18, and IFNβ Release in Murine Macrophages.

Talabostat mesylate (MedChemExpress; TLBST) was reconstituted to 10 mg/mL in deionized water and stored frozen. Prior to use, TLBST was thawed, incubated at 37° C. for 20 mins, and sonicated for 10 minutes. TLBST was serially diluted in pH 7.4, 40 mM Tris, 10 mM MgCl₂ buffer to 5 mg/mL and 2.5 mg/mL. Free talabostat controls were prepared in triplicate by diluting and serially diluting the 10, 5, and 2.5 mg/mL TLBST stock in pH 7.4, 40 mM Tris-HCl, 10 mM MgCl₂ buffer to generate free TLBST at 1.25, 0.625, and 0.3125 mg/mL. DNAO controls (0 mg/mL TLBST) were prepared in triplicate by diluting a 144.5 nM purified DNAO stock in TE with 20 mM MgCl₂ buffer and pH 7.4, 40 mM Tris-HCl, 10 mM MgCl₂ buffer to a 20 nM concentration. 20 nM DNAO was loaded with TLBST in triplicate by incubating DNAO with TLBST at 1.25, 0.625, and 0.3125 mg/mL. DNAO samples loaded with 0-1.25 mg/mL TLBST and free TLBST controls at 0.3125-1.25 mg/mL were incubated on a stir table for 2 hours at 150 rpm.

RAW264.7 cells were seeded at 100,000 cells per well of a 96-well plate. Twenty-four hours later, cells were stimulated by addition of 20 μL of the indicated material to 80 μL cells in growth medium. Vehicle represents Tris TE buffer; DNAO represents 9 nM cuboid DNAO (assembled using a 7560-nucleotide M13mp18 scaffold from tilibit nanosystems) in TE buffer; 0.9 mM free TLBST represents talabostat mesylate (MedChemExpress) reconstituted in deionized water, stored frozen, and diluted in Tris buffer; and DNAO loaded in 1 mM TLBST represents cuboid DNAO incubated in a solution of 1 mM talabostat mesylate for 2 hours with shaking prior to an ultracentrifugation-based 100 kDa cutoff purification and dilution in Tris TE buffer. Twenty-four hours following stimulation, cell culture supernatants were harvested and stored frozen until quantification of lactate dehydrogenase (LDH) by colorimetric enzymatic activity assay, and interleukin 1β (IL-1β), interleukin 18 (IL-18), and interferon β (IFNβ) by multiplex cytometric bead array assay. Percentage LDH release was calculated relative to the maximum LDH released from a lysed cells control. Biomarker concentrations were interpolated from reference standard curves for each analyte. MTT signal derived from stimulated cells was quantified by colorimetric enzymatic activity assay. Percentage viability was calculated relative to the MTT signal measured in an untreated cells control. All enzymatic activity and biomarker assays were carried out using commercially available kits in accordance with manufacturer's recommendations.

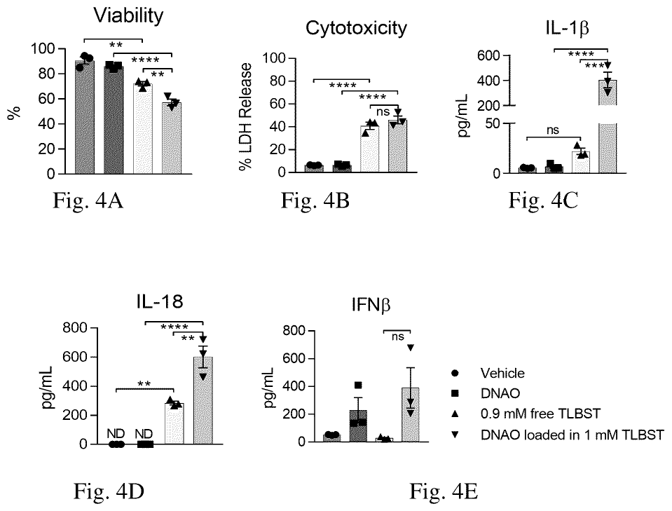

FIG. 4 shows the effect of DNAO-TLBST on cytotoxicity and cytokine release in murine macrophages. The results in murine macrophages treated with non-functionalized DNAO-TLBST suggest that these nanocarriers deliver and release TLBST in cells, leading to pyroptosis induction.

### Example 5

Impact of CTP-DNAP-TLBST on Human Myeloid Cells In Vitro

Pyroptosis is characterized by inflammasome activation, caspase 1-mediated IL-1β and IL-18 maturation, and the release of pro-inflammatory cellular contents through plasma membrane pores and cell lysis. Systemic administration of pyroptosis-inducing small molecules for cancer treatment is an area of active investigation. The inventors have shown that DNAO-TLBST recapitulated the effect of free TLBST on murine macrophage cytotoxicity and LDH release (FIG. 4) indicating DNAO delivers biologically active TLBST to cells. In this example, we will test the cytotoxicity and immunogenicity of IL4RPep-1-functionalized DNAO-TLBST (CTP-DNAO-TLBST) on human macrophage cytotoxicity.

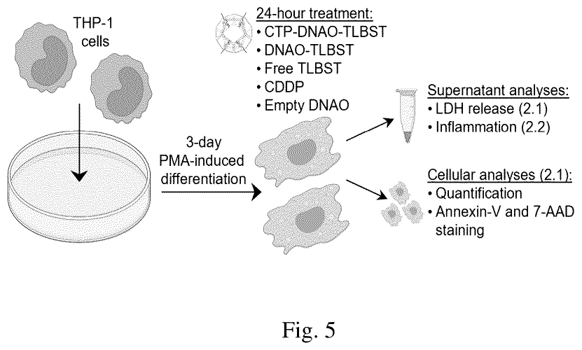

We will evaluate the impact of CTP-DNAO-TLBST as described in Example 3 on human macrophage cytotoxicity in vitro, to avoid differential cell death responses and crosstalk between heterogeneous cell types. Differentiated THP-1 cells will be treated as illustrated in FIG. 5. After 24 hours of stimulation, supernatants and cells will be harvested. A portion of each supernatant sample will be used to assay cytotoxicity via a colorimetric enzymatic assay measuring lactate dehydrogenase (LDH). Cells harvested from each fraction will be used to analyze cellular viability. We will detect exposed phosphatidylserline (PS; a feature of apoptotic cells) by Annexin V staining. Cell membrane integrity will be assessed using the fluorescent DNA-binding dye 7-AAD. Staining and subsequent flow cytometric acquisition will be performed in a 96-well plate format to increase throughput and to enable quantification of cellularity using a syringe-drawn acquisition system. Non-apoptotic cytotoxicity would be indicated by decreased viable (Annexin V⁻ 7-AAD⁻) cells, increased LDH release, and a lack of Annexin V cells. These features may or may not be accompanied by an increase in dead (7-AAD⁺) cells, depending upon the kinetics of cell death. Apoptotic cytotoxicity would be indicated by decreased viable cells and increased Annexin V⁺ cells, with or without increased dead cells and LDH release. We predict that CTP-DNAO-TLBST will induce greater cytotoxicity compared to non-functionalized DNAO-TLBST. Additionally, we expect that CTP-DNAO-TLBST-induced cytotoxicity will be consistent with non-apoptotic cell death (Table 1).

Apoptotic cells present and release anti-inflammatory and regenerative mediators including prostaglandin E₂ (PGE2), transforming growth factor beta (TGFβ), and IL-1β, which promote cellular proliferation and immune-suppression. In addition to cytotoxicity, our analyses demonstrate IL-1β and IL-18 release from DNAO-TLBST-treated murine macrophages (FIG. 4), indicating that DNAO-TLBST induces macrophage pyroptosis. IL-1β and IL-18 promote dendritic cell (DC) maturation and antigen presentation, thus driving T helper type 1 CD4⁺ and CD8⁺ T cell responses, interferon gamma (IFNγ) production, tumor antigen presentation, and generation of anti-tumor immune responses.

In addition to delivering the TLBST payload to target cells, DNAO is an intrinsic activator of endosomal and cytosolic DNA sensors. Upon activation, these sensors establish an antiviral immune program characterized by type I interferon (IFNα and IFNβ) expression. Thus, DNAO encapsulation acts as an adjuvant likely to accelerate T cell-mediated immunity. Indeed, DNAO-TLBST but not free TLBST is associated with IFNβ secretion from murine macrophages (FIG. 4). Using supernatant from the same samples outlined above in this Example, we will profile the inflammatory secretome of human macrophages following CTP-DNAO-TLBST and control treatment using multiplex bead-based immunoassays. This panel will include type I IFNs, IFNγ, IL-1α, IL-1β, IL-10, IL-18, MCP-1/CCL2, TNFα, IL-6, and IL-12p70. We will quantify each analyte concentration independently, and will examine relationships between pro- and anti-inflammatory mediators as ratios of each pro-inflammatory factor to IL-10. We predict CTP-DNAO-TLBST will induce secretion of the primary pro-inflammatory endpoints: type I IFNs, IL-1β, and IL-18, with potential increases in the secondary pro-inflammatory endpoints: IFNγ, MCP-1/CCL2, TNFα, IL-6, and IL-12p70, and little to no induction of the anti-inflammatory endpoint IL-10. We expect a similar profile with non-functionalized TLBST-DNAO, though lower in magnitude due to reduction in the extent or rate of cellular uptake. We anticipate relatively specific impacts of free TLBST on IL-1β and IL-18, and DNAO on type 1 IFNs (Table 2).

Tables 1 and 2

| | LDH | Viable | | |
|---|---|---|---|---|
| Condition | release | Cells | Annexin-V⁺ | 7-AAD⁺ |
| Control | — | ↑↑↑ | — | — |
| CTP-DNAO-TLBST | ↑↑↑ | ↓↓↓ | — | ↑↑↑ |
| DNAO-TLBST | ↑↑ | ↓↓ | — | ↑↑ |
| Free TLBST | ↑↑ | ↓ | — | ↑ |
| CDDP | ↑ | ↓ | ↑ | ↑ |
| DNAO | — | ↑↑↑ | — | — |

| | Type I | IL-1β, | |
|---|---|---|---|
| Condition | IFNS | IL-18 | IL-10 |
| Control | — | — | — |
| CTP-DNAO-TLBST | ↑↑↑ | ↑↑↑ | — |
| DNAO-TLBST | ↑↑ | ↑↑ | — |
| Free TLBST | — | ↑↑↑ | — |
| CDDP | — | — | ↑↑ |
| DNAO | ↑↑ | — | — |

### Example 6

DNAO-TLBST Induces Cytotoxicity and Concomitant IL-1β, IL-18, and IFNβ Release in Human Macrophages.

Talabostat mesylate (MedChemExpress; TLBST) was reconstituted to 10 mg/mL in pH 7.4, 40 mM Tris, 10 mM MgCl₂ buffer. Prior to use, TLBST was incubated at 37° C. for 20 mins, and sonicated for 10 minutes. TLBST was serially diluted in pH 7.4, 40 mM Tris, 10 mM MgCl₂ buffer to 5 mg/mL and 2.5 mg/mL. Free TLBST controls were prepared by diluting the 10, 5, and 2.5 mg/mL stock solution and serial dilutions in pH 7.4, 40 mM Tris-HCl, 10 mM MgCl₂ buffer to generate free TLBST at 1.25, 0.625, and 0.3125 mg/mL. DNAO controls with 0 mg/mL of talabostat were prepared by diluting 162 nM purified DNAO stock in TE with 20 mM MgCl₂ buffer and pH 7.4, 40 mM Tris-HCl, 10 mM MgCl₂ buffer to a 20 nM concentration. DNAO loading was carried out in duplicate by incubating 20 nM DNAO in 1.25, 0.625, and 0.3125 mg/mL TLBST. DNAO samples loaded with 0-1.25 mg/mL TLBST and free TLBST controls at 0.3125-1.25 mg/mL were incubated on a stir table for 2 hours at 150 rpm.

THP-1 cells seeded at 100,000 cells per well of a 96-well plate were induced to undergo macrophage differentiation through administration of 20 ng/mL phorbol myristate acetate (PMA). Differentiation in the presence of PMA was allowed to proceed for three days prior to removal of PMA containing media and replacement with normal growth media. Three days later, cells were stimulated by addition of 20 μL of the indicated material to 80 μL cells in growth medium. Vehicle represents Tris TE buffer; DNAO represents 9 nM cuboid DNAO (assembled using a 7560-nucleotide M13mp18 scaffold from tilibit nanosystems) in TE buffer; 1 mM free TLBST represents talabostat mesylate (MedChemExpress) reconstituted in deionized water and diluted in Tris buffer; and DNAO loaded in 1 mM TLBST represents cuboid DNAO incubated in a solution of 1 mM talabostat mesylate for 2 hours with shaking prior to an ultracentrifugation-based 100 kDa cutoff purification and dilution in Tris TE buffer. Twenty-four hours following stimulation, cell culture supernatants were harvested, cleared by centrifugation, and stored frozen until quantification of lactate dehydrogenase (LDH) by colorimetric enzymatic activity assay, and interleukin 13 (IL-1β), interleukin 18 (IL-18), and interferon β (IFNβ) by multiplex cytometric bead array assay. Percentage LDH release was calculated relative to the maximum LDH released from a lysed cells control. Biomarker concentrations were interpolated from reference standard curves for each analyte. LDH enzymatic activity and biomarker assays were carried out using commercially available kits in accordance with manufacturer's recommendations.

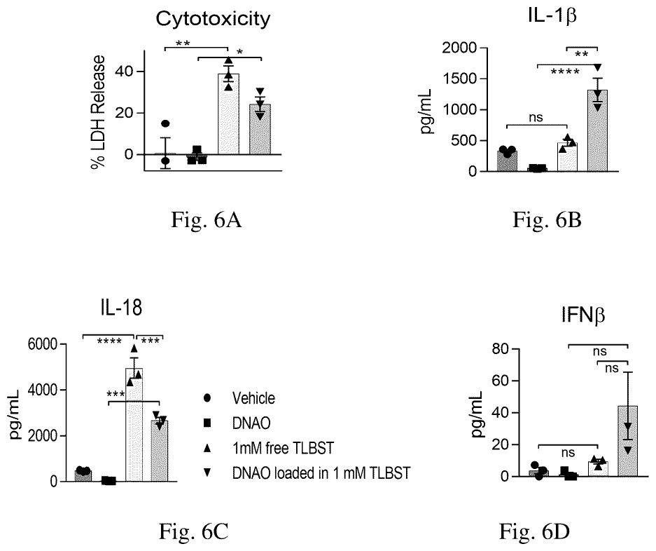

FIG. 6 shows the effect of DNAO-TLBST on cytotoxicity and cytokine release in human macrophages. The results in human macrophages treated with non-functionalized DNAO-TLBST suggest that these nanocarriers deliver and release TLBST in cells, leading to pyroptosis induction.

### Example 7

DNAO-TLBST Induces Cytotoxicity and Concomitant IL-1β, IL-18, and IFNβ Release in Human Prostate Epithelial Cells.

Talabostat mesylate (MedChemExpress; TLBST) was reconstituted to 10 mg/mL in deionized water and stored frozen. Prior to use, TLBST was thawed, incubated at 37° C. for 20 minutes, and sonicated for 10 minutes. TLBST was serially diluted in pH 7.4, 40 mM Tris, 10 mM MgCl₂ buffer to 5 mg/mL and 2.5 mg/mL. Free TLBST controls were prepared in triplicate by diluting the 10, 5, and 2.5 mg/mL TLBST stock and serial dilutions in pH 7.4, 40 mM Tris-HCl, 10 mM MgCl₂ buffer to generate 1.25, 0.625, and 0.3125 mg/mL solutions. DNAO controls with 0 mg/mL TLBST were prepared in triplicate by diluting a 143 nM purified DNAO stock in TE with 20 mM MgCl₂ buffer and pH 7.4, 40 mM Tris-HCl, 10 mM MgCl₂ buffer to a 20 nM concentration. DNAO was loaded in triplicate reactions by incubation of 20 nM DNAO in 1.25, 0.625, and 0.3125 mg/mL TLBST. DNAO samples loaded with 0-1.25 mg/mL TLBST and free TLBST controls at 0.3125-1.25 mg/mL were then incubated on a stir table for 2 hours at 150 rpm.

PC-3 cells were seeded at 10,000 cells per well of a 96-well plate twenty-four hours prior to stimulation by the addition of 20 μL of the indicated material to 80 μL cells in growth medium. Vehicle represents Tris TE buffer; DNAO represents 9 nM cuboid DNAO (assembled using a 7560-nucleotide M13mp18 scaffold from tilibit nanosystems) in TE buffer; 0.9 mM free TLBST represents talabostat mesylate (MedChemExpress) reconstituted in deionized water and diluted in Tris buffer; and DNAO loaded in 1 mM TLBST represents cuboid DNAO incubated in a solution of 1 mM talabostat mesylate for 2 hours with shaking prior to an ultracentrifugation-based 100 kDa cutoff purification and dilution in Tris TE buffer. Twenty-four hours following stimulation, cell culture supernatants were harvested, cleared by centrifugation, and stored frozen until quantification of lactate dehydrogenase (LDH) by colorimetric enzymatic activity assay performed according to manufacturer's recommendations. Percentage LDH release was calculated relative to the maximum LDH released from a lysed cells control.

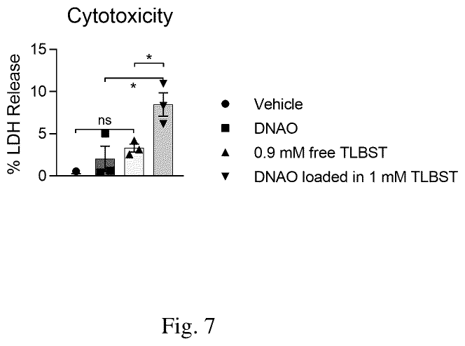

FIG. 7 shows the effect of DNAO-TLBST on cytotoxicity and cytokine release in human prostate epithelial cells. The results in human prostate epithelial cells treated with non-functionalized DNAO-TLBST suggest that these nanocarriers deliver and release TLBST in cells, leading to pyroptosis induction.

What is claimed is:

1. A composition comprising a non-viral delivery vehicle comprising a nucleic acid nanostructure delivery composition, and a small molecule therapeutic.

2. The composition of claim 1, wherein the nucleic acid nanostructure delivery composition comprises a DNA origami composition.

3. The composition of claim 1, wherein the nucleic acid nanostructure delivery composition comprises single-stranded or double-stranded DNA or RNA.

4. The composition of claim 2 wherein the nucleic acid nanostructure delivery composition comprises both single-stranded and double-stranded regions of the nucleic acids.

5. The composition of claim 3 wherein the nucleic acid nanostructure delivery composition is single-stranded.

6. The composition of claim 3 wherein the nucleic acid nanostructure delivery composition is double-stranded.

7. The composition of claim 1, wherein the small molecule therapeutic is bound to the nucleic acid nanostructure delivery composition by a covalent bond.

8. The composition of claim 7, wherein the covalent bond is formed via an EDC-NHS coupling reaction between a terminal phosphate group of the 5′ end of an overhang on the nucleic acid nanostructure delivery composition and an amine group on the small molecule therapeutic.

9. The composition of claim 7, wherein the covalent bond is formed via a click chemistry coupling reaction between an azide group on the nucleic acid nanostructure delivery composition and an alkyne group on the small molecule therapeutic.

10. The composition of claim 7, wherein the covalent bond is formed via a click chemistry coupling reaction between an azide group on the small molecule therapeutic and an alkyne group on the nucleic acid nanostructure delivery composition.

11. The composition of claim 1, wherein the small molecule therapeutic is associated with the nucleic acid nanostructure delivery composition by a covalent bond between a carboxy terminated molecule on the nucleic acid nanostructure delivery composition and a primary amine on the small molecule therapeutic.

12. The composition of claim 1, wherein the small molecule therapeutic is associated with the nucleic acid nanostructure delivery composition by an electrostatic interaction between a negatively charged nucleic acid nanostructure delivery composition and a positively charged amine in the small molecule therapeutic.

13. The composition of claim 1, wherein the small molecule therapeutic is associated with the nucleic acid nanostructure delivery composition by intercalation of the small molecule therapeutic into the nucleic acid nanostructure delivery composition.

14. The composition of claim 1, wherein the small molecule therapeutic is associated with the nucleic acid nanostructure delivery composition by base pairing wherein the small molecule therapeutic comprises a nucleic acid covalently bound to the small molecule therapeutic and wherein the nucleic acid covalently bound to the small molecule therapeutic is base-paired to a complementary nucleic acid on the nucleic acid nanostructure delivery composition.

15. The composition of claim 1, wherein the small molecule therapeutic causes pyroptosis.

16. The composition of claim 1, wherein the small molecule therapeutic induces anti-tumor immunity.

17. The composition of claim 1, wherein the small molecule therapeutic induces cytokine production.

18. The composition of claim 1, wherein the small molecule therapeutic induces inflammasome activation.

19. The composition of claim 1, wherein the small molecule therapeutic induces the production of an interferon or an interleukin.

20. The composition of claim 19, wherein the interferon and the interleukin are selected from a type one interferon, IFN-β, IFN-γ, IL-1β, IL-6, IL-12p70, and IL-18.

21. The composition of claim 1, wherein the small molecule therapeutic is talabostat.

22. The composition of claim 1, wherein the nucleic acid nanostructure delivery composition is coated with one or more polymers.

23. The composition of claim 22, wherein the one or more polymers comprise PEG-poly-L-lysine.

24. A method of treating a patient with a disease, the method comprising administering to the patient the composition of claim 1, and treating the disease in the patient.

25. The method of claim 24, wherein the nucleic acid nanostructure delivery composition is not cytotoxic to the cells of the patient.

26. The method of claim 24, wherein the small molecule therapeutic induces anti-tumor immunity by targeting immunosuppressive cells.

27. The method of claim 26 wherein the immunosuppressive cells are selected from tumor-associated macrophages and myeloid-derived suppressor cells.

28. A method of treating a patient with a disease, the method comprising administering to the patient the composition of claim 14, and treating the disease in the patient.

29. A method of treating a patient with a disease, the method comprising administering to the patient the composition of claim 21, and treating the disease in the patient.

30. The method of claim 29 wherein the nucleic acid nanostructure delivery composition and the small molecule therapeutic both cause anti-tumor immunity in the patient.
